(19) United States
(12) Patent Application Publication
(10) Pub. No.: US 2003/0119768 A1
(43) Pub. Date: Jun. 26, 2003

(54) Method for use of oligonucleotide therapeutics

(76) Inventors: Thomas D. Madden, Vancouver (CA); Murray S. Webb, Surrey (CA)

Correspondence Address: OPPEDAHL AND LARSON LLP, P O BOX 5068, DILLON, CO, 80435-5068, US

(21) Appl. No.: 10/012,010

(22) Filed: Oct. 29, 2001

Related U.S. Application Data
(60) Provisional application No. 60/245,176, filed on Nov. 2, 2000.

(51) Int. Cl. A61K 48/00; A61K 9/16; A61K 9/50

(52) U.S. Cl. 514/44; 424/493

(57) ABSTRACT

It has now been discovered that oligonucleotides which tend to form multimeric aggregates have greater toxicity in the aggregate form than in monomeric form. Thus, administration of a therapeutic oligonucleotide that tends to form multimeric aggregates is suitably preceding by a treatment to convert substantially all of the therapeutic oligonucleotide to a monomeric form or to substantially prevent the formation of multimeric aggregates; and then administering the composition in which substantially all of the therapeutic oligonucleotide is in monomeric form to a mammal in need of therapy provided by the oligonucleotide. The composition can be treated by heating, preferably no more than 24 hours prior to administration, or using chemical species such as mannitol or sucrose which disrupt aggregates.

This application claims the benefit of U.S. Provisional Application No. 60/245,176, filed Nov. 2, 2000 which is incorporated herein by reference.

### BACKGROUND OF THE INVENTION

This application relates to an improved method for use of oligonucleotide therapeutics which reduces the toxicity, thus allowing for the use of higher and more efficacious doses.

Short (5-50 mer) oligonucleotide compositions have demonstrated some promise as therapeutic agents in the clinic. The mechanism of activity is asserted by some to be through mRNA binding (or “antisense”) effects; although others note that therapeutic results in animal models are often consistent with an immune system stimulation mechanism which is independent of an antisense mechanism. Regardless of the mechanism, performance has been below expectation. One important reason for product failures has been dose limiting toxicities identified in in vivo trials.

Toxicity of oligonucleotides has historically been attributed to chemical modifications made to the compositions prior to patient administration. Naturally occurring oligonucleotides are generally unsatisfactory for use, in the free form, because the phosphodiester (“PO”) linkages are highly susceptible to degradation in the blood. Natural oligonucleotides in the free form do not circulate in mammals long enough to accumulate at sites of disease. Chemical modifications are introduced primarily to reduce susceptibility to blood borne exo- and endo-nucleases.

Many modifying chemistries tried to date have a higher degree of toxicity in standard toxicity assays than PO oligonucleotides. For phosphorothioates (PS), both in vitro assays of complement activation and coagulation (Levin et al., in Handbook of Experimental Pharmacology, G V R Born et al., eds, Springer-Verlag, Berlin, pp. 169-215 (1998)) and in vivo assays of toxicity, such as liver enzyme release, etc., are increased relative to POS, (Henry, S P, et al., J Pharmacol. Exp. Ther. 281: 810-816 (1997)). A transient increase in activated partial thromboplastin tie (APTT) is a well-characterized phosphorothioate class-effect (Levin et al. (1998)). (see Sheeham, J P. and Lan H C, Blood, 92:1617 (1998) and Henry, S P, Antisense Nucleic Acid Drug Dev. 7:503 (1997)). In vivo experiments have been complicated by alleged species specific toxicity responses which have confounded the traditional use of toxicity models for predicting toxicity in other species. Monetieth et al., Anti-Cancer Drug Design 12: 421-432 (1997).

Other sources of toxicity of oligonucleotides have not been carefully investigated. It is an object of the instant invention to provide oligonucleotide compositions which demonstrate a surprisingly and substantially reduced toxicity profile upon administration to a mammal. In addition, the invention provides methods of reducing the toxicity of oligonucleotides for use as therapeutic agents.

### SUMMARY OF THE INVENTION

It has now been discovered that oligonucleotides which tend to form multimeric aggregates have greater toxicity in the aggregate form than in monomeric form. Thus, the present invention provides a method for administration of a therapeutic oligonucleotide that tends to form multimeric aggregates comprising the steps of:

(a) treating a composition comprising the therapeutic oligonucleotide in multimeric aggregate form to convert substantially all of the therapeutic oligonucleotide to a monomeric form or to substantially prevent the formation of multimeric aggregates; and

(b) administering the composition in which substantially all of the therapeutic oligonucleotide is in monomeric form to a mammal in need of therapy provided by the oligonucleotide. The composition can be treated by heating, preferably no more than 24 hours prior to administration, or using chemical species such as mannitol which disrupt aggregates.

### BRIEF DESCRIPTION OF THE DRAWINGS

FIGS. 1A and B show the in vivo metabolism of INX-3280 (Seq. ID No. 1) in cynomolygus monkeys; and

FIG. 2 shows the affect of monomerization on coagulation in vitro.

### DETAILED DESCRIPTION OF THE INVENTION

The present application relates to method for reducing the toxicity of therapeutic oligonucleotide compositions and to the use of the resulting reduced-toxicity compositions for providing therapeutic benefits to mammals, including humans. A common feature of the oligonucleotides to which the invention relates is the spontaneous formation of multimeric aggregates. There are many known configurations for base pairing which can lead to the formation of such multimers, including the well-known Watson and Crick-type of base pairing, Hoogsteen base pairing (a somewhat weaker association of a purine and a pyrimidine through the formation of a different hydrogen bonds) and forms of various combinations of hetero (purine+pyrimidine) and homo (purine+purine or pyrimidine+pyrimidine) base pairs. Thus, interactions may occur to lead to the multimerization in oligonucleotides with a variety of structures.

The present invention is based upon the discovery that multimeric oligonucleotide aggregates are associated with toxicity when oligonucleotides are administered, and that the overall toxicity of a therapeutic oligonucleotide composition can be reduced by treating the composition to convert substantially all of the multimers present into monomers or to substantially prevent the formation of multimeric aggregates. Because the interactions between the oligonucleotides in the aggregates are relatively weak hydrogen bonding interactions, the aggregates and the monomers exist in solution in an equilibrium. This means that at any given set of conditions, some mixture of aggregates and monomers is present. For example, in a conventional (untreated) solution of the oligonucleotide INX-3280 (described below), which tends to form multimeric aggregates, the ratio (by weight or by mole) of oligonucleotide molecules found in monomers versus tetrameric complexes is about 40-60:60-40. The goal of the present invention is to disrupt this equilibrium prior to use of the oligonucleotide in treatment so that substantially all of the oligonucleotide is present in monomeric form. As used in the specification and claims of this application, the term “substantially all” refers to levels of monomeric oligonucleotide in excess of 75% by weight, preferably in excess of 95% by weight. A procedure which substantially prevents the formation of multimeric complexes is one which is effective to prevent formation of multimeric complexes amounting to more than about 25% by weight of the total oligonucleotide present.

In one embodiment of the invention, an oligonucleotide composition is treated prior to administration by heating the composition to a temperature and for a period of time sufficient to convert aggregates to monomers without damaging the oligonucleotide. In general, the composition is suitably heated to a temperature of 45 to 100° C. for 3 to 180 minutes. For clinical use, sample are preferably treated at 60° C. for 30 minutes or 90° C. for 3 minutes. The optimum time and temperature required for a particular composition depends on the strength of the hydrogen bonding in the multimers, as well as other factors, but can be readily determined for any particular oligonucleotide by assaying for levels of monomers and multimers or by assaying for toxicity reduction. Procedures for such assays are known in the art, and are described in the examples set forth below.

In an alternative embodiment of the invention, a chemical additive that is effective to prevent formation of the hydrogen bonds of the aggregate is introduced into the composition. This additive should be pharmaceutically acceptable, such that it does not itself induce unfavorable reactions upon in vivo administration. One example of a suitable compound capable of maintaining newly synthesized oligos (which have not yet aggregated to multimeric form) or heat treated oligos which have been converted to substantially monomeric form as monomers is mannitol. Mannitol is thought to act during the lyophilization step, where multimeric aggregates tend to form. Use of mannitol result in a monomeric form of the oligonucleotide upon reconstitution in water or a pharmaceutically-acceptable buffer. Mannitol may be used at concentrations of from 50 to 500 mM. Other compounds which might be used for this purpose include other mono-, di- or oligosaccharides such as sucrose (10-1000 mM), glucose, trialose or lactose etc., and pharmaceutically acceptable cationic species such as Mn⁺, Co⁺, Ni²⁺ and Tb (III).

The therapeutic oligonucleotides to which the invention is applicable generally have a size of 5 to 50 bases. The oligonucleotides may provide therapeutic benefit through an antisense effect, i.e, by binding to complementary sequences in RNA and inhibiting protein synthesis, or they may provide therapeutic benefit through a non-sequence specific method. Thus, the methods of the invention may be used to provide therapeutic benefits in connection with the treatment of a wide variety of diseases and conditions.

One specific, non-limiting example of oligonucleotides which undergo multimerization are those which include a sequence of 4 G residues. Such oligonucleotides aggregate into a quadraplex. For example, a 15-mer directed against c-myc RNA, sometimes caled INX-3280, is known and has the sequence:

AAC GTT GAG GGG CAT Seq. ID. No. 1

Prior to the invention disclosed herein, the phosphorothioate version of this molecule, (which is sufficiently stable for direct administration), had been shown to suffer from the same toxicities as the other members of the phosphorothioate class (Henry et al. 1997a; Henry et al., 1997b; Levin et al. 1998; Monteith et al., 1998). Phosphorothioate oligonucleotides have a well recognized class effect in monkeys that can lead to life-threatening hemodynamic disturbances (Galbraith et al., 1994; Henry et al., 1997a; Henry et al., 1997b; Monteith et al., 1998). Fortunately, this effect is blood-level dependent and is usually ameliorated by diminishing the rate of intravenous infusion (i.e., by increasing the duration of infusion to deliver the desired dose). However, the threshold concentration for complement activation of 40-50 μg/mL is generally reported for phosphorothioate oligonucleotides (Henry et al., 1997a; Henry et al., 1997b; Monteith et al., 1998; Levin et al., 1998).

In contrast, as illustrated in the examples below, when INX-3280 antisense oligodeoxynucleotide is administered after treatments which leave the compound in monomeric form, the toxicity decreases dramatically. In vivo doses having peak INX-3280 concentrations in the plasma of 101.5 to 119.6 μg/mL in monkeys treated at 15 mg/kg were associated with no significant increases in Bb values (×1 μg/mL) and only minor (30%) increases in APTT values (see Examples).

Other known or proposed therapeutic oligonucleotides which include a 4-G motif (i.e. a sequence of four consecutive G residues) include, but are not limited to, certain c-myb and RelA (targeting the p65 subunit of NF-kappaB) sequences, etc. These compounds have been demonstrated to form multimeric aggregates, based on what is likely an anti-parallel array based on an arrangement of G residues. Other oligonucleotides that do not contain 4G motifs may also form complex multimers, either by specific or non-specific binding arrangements. These include particularly oligonucleotides with palindrome or partial palindrome sequences. The therapeutic benefit of all these oligonucleotides can be enhanced by the methods taught in the instant invention.

In accordance with the invention, the oligonucleotides are administered directly to a subject in need of therapy. Preferred modes of administration are by injection, and include intravenous, intraparenteral, intramuscular and/or subcutaneous injections. Whenever the oligonucleotides are introduced directly into the circulation, they are at risk for degradation by nucleases. Thus, the invention preferably makes use of oligonucleotides which have been structurally modified to increase their resistance to nucleases, where the modifying chemistry does not itself interfere with the formation of multimeric complexes. This includes phosphorothioate (PS) oligonucleotides, 2′-O-methyl oligonucleotides, methoxy-ethoxy oligonucleotides, chimerics and gap-mers DNA/RNA hybrids, phosphorodithioate oligonucleotides, morpholino oligonucleotides and amidate-derivatives of oligonucleotides. The oligonucleotide composition is administered in a pharmaceutically acceptable liquid carrier appropriate for administration of injectable therapeutics. This would include sterile saline solution, 5% dextrose in water, phosphate buffered saline (PBS) and the like.

It will be appreciated that because the aggregation of oligonucleotides is a spontaneous event, that the disruption of the equilibrium by heating may not be permanent, and that some steps may be required to achieve the desired level of monomers at the time of administration. When heating is used to disrupt to dissociate the multimers, a simple expedient is perform the heating step within a short time prior to administration. For example, in the case of 4G oligonucleotides such as c-myc, the time required for a return the equilibrium mixture of monomer and tetramer is several days. Thus, the benefits of the invention can be obtained if the composition is used within 24 hours of heating. Alternatively, a chemical additive ((such as mannitol or sucrose) can be added to the heated material to substantially prevent the reformation of monomers.

As illustrated in the examples below, the monomer structure is retained upon lyophilization of cryoprotectant-treated compositions. Thus, compositions which have been converted to substantially monomeric form may be lyophilized in the presence of a cryoprotectant such as sucrose or mannitol and reconstituted just prior to administration. In the resulting reconstituted composition, substantially all of the oligonucleotide is in the monomeric form. Of course, where reversion to the equilibrium mix of monomers and multimers is an issue, this reconstituted composition must be used promptly.

Using the method of the invention, several new therapeutic approaches to disease treatment are suggested. In particular, the treatments that monomerize oligonucleotides will be useful for combination treatments that use other chemotherapeutic agents. Cisplatin and other metal based chemotherapeutics are particularly advantageous in combination therapy. Thus this invention opens up significant new treatment options for using chemotherapeutic agents in clinical indicaitons for which they are not currently recommended. In particular, the monomerized oligonucleotides of the invention now enable the therapeutic use of cisplatin, in combination with oligonucleotides of the invention, for the treatment of disorders, particularly cancers, and more particularly cancers of the head and neck, upper gastro-intestinal cancers such as hepatocarcinoma or cancers of the lower or upper oesophagus, and metastatic breast cancer.

The invention will now be further described with reference to the following, non-limiting examples.

### EXAMPLE 1

The phosphorothioate oligonucleotide INX-3280 was manufactured according to current Good Manufacturing Practices using methods described previously (Fearon et al., 1997). INX-3280 was lyophilized in 20-mg vial by standard methods and stored at 2-8° C. INX-3280 is a 15-mer sequence directed against the c-myc mRNA having the sequence:

INX-3280: 5′-AAC GTT GAG GGG CAT-3′ Seq. ID. No. 1

Each 20-mg vial of lyophilized INX-3280 was reconstituted with 4 mL of sterile saline. These solutions were placed in a water bath at 60-70° C. for 30 minutes to convert the oligonucleotide to its monomeric form. After cooling, the solutions were diluted with sterile saline to the appropriate concentrations (approximately 0.5 and 2.5 mg/mL) for administration.

For the in vitro assays described below (Example 3), INX-3280 was used before and after the heating (monomerization) process. The proportion of INX-3280 in the monomeric and quadruplex forms was quantified by Size Exclusion Chromatograph-HPLC (SEC-HPLC) on a Pharmacia Superdex 75HR 10/30 gel filtration column. An isocratic gradient of 25 mM sodium phosphate buffer with 0.25 mM Na₂EDTA (pH 7.5) was used at a flow rate of 0.75 ml/minute at room temperature. Oligonucleotide was detected by absorbance at 260 nm. As summarized in Table 1, After initial hydration in saline, the unmonomerized INX-3280 was present as a 46:54 mixture of the monomeric and quadruplex forms. Heating at 60-70° C. for 30 minutes effectively dissociated the quadruplex and yielded a preparation comprised of >98% monomeric oligonucleotide. The monomeric form of INX-3280 was stable for at least 24 hours when held at room temperature.

TABLE 1

| | Proportion (% of total AUC) | | |
|---|---|---|---|
| Treatment | Monomer | Quadruplex | Other* |
| Reconstituted in saline (unmono- | 46.0 | 54.0 | 0 |
| merized) | | | |
| Reconstituted in saline + 60-70° C. | 98.2 | 1.6 | 0.1 |
| for 30 min | | | |
| Reconstituted in saline + 60-70° C. | 99.2 | 0.8 | 0 |
| for 30 min + 24 h at room tempera- | | | |
| ture | | | |

*higher order aggregates.

### EXAMPLE 2

The selection of Cynomolgus monkeys for in vivo testing and design of this study was based on previous experience with development of other phosphorothioate oligonucleotides, and with the published guidelines for development of drugs in this class (Black et al., 1994; Ahn and DeGeorge, 1998).

Sixteen (eight male and eight female) experimentally naive cynomolgus monkeys (Macaca fascicularis) were obtained from Scientific Resources International (Reno, Nev.). Animals had pre-study mean weights of 2.5 to 3.0 kg. The animals were housed individually in stainless-steel cages on a 12-hour light/12-hour dark photoperiod at 18 to 28° C. Animals were provided with weight-appropriate amounts of Harlan Teklad Primate Diet as well as with supplemental fruits, vegetables and cereal. Water was provided ad libitum. Treatment of animals was in accordance with the regulations defined in the USDA Animal Welfare Act (9 CFR Parts 1, 2 and 3).

Animals were assigned to groups by a stratified randomization scheme designed to achieve similar group mean body weights; then the groups were randomly assigned to treatments. Group 1 (two males and two females) were treated exclusively with sterile saline. Group 2 (three males and three females) were treated with monomeric INX-3280 at a dose of 3.0 mg/kg. Group 3 (three males and three females) were treated with monomeric INX-3280 at a dose of 15.0 mg/kg. Doses were administered by intravenous infusion on days 1, 3, 5, 8, 10 and 12. A venous catheter was inserted into a cephalic or saphenous vein, and an infusion or piston pump was used to deliver the saline (control) or oligonucleotide dose at a controlled rate of 3.0 mL/kg/h for a 2 h period.

Clinical Signs and Physiologic Measurements

Animals were monitored twice daily for clinical signs (general appearance, behavior, food consumption). Blood pressure and heart rate were measured with an external cuff instrument (Dynamap) prior to and at the end of the first and last infusions (Days 1 and 12) from an arm or leg. Systolic/diastolic pressure, mean arterial pressure (MAP), and heart rate were recorded. Body weights were measured seven and three days prior to the initiation of the study and again at the end of the study.

Hematology

Blood samples (0.5 mL) were collected in EDTA-containing tubes pre-infusion, at the end of infusion and 4 h post-infusion after the first dose (day 1) and the last dose (day 12). Whole blood was analyzed for red blood cell (RBC) counts, white blood cells (WBCs; total and differential), hemoglobin concentration, hematocrit, mean cell hemoglobin (MCH), mean corpuscular volume (MCV), mean corpuscular hemoglobin concentration (MCHC), platelet counts and blood cell morphology. In addition, reticulocyte counts were determined prior to the first dose (baseline) and 24 h post-infusion after the last dose (day 13).

Serum Chemistry

Blood samples (1.5 mL) were collected into tubes without anticoagulant, and the blood was allowed to clot and centrifuged to obtain serum. Blood samples for serum chemistry were obtained pre-infusion on the first dosing day (day 1) and 24 h after the last dose (day 13). Sera obtained from these samples were analyzed for the concentrations of sodium, potassium, chloride, total carbon dioxide (bicarbonate), total bilirubin, calcium, phosphorous, glucose, urea nitrogen (BUN), creatinine, total protein, albumin, globulin, cholesterol and triglycerides as well as for the activities of alkaline phosphatase (AP), lactate dehydrogenase (LDH), aspartate aminotransferase (AST), alanine aminotransferase (ALT) and gamma-glutamyltransferase (GGT).

Coagulation and Complement Assays

For the assessment of coagulation parameters, approximately 0.9 mL of blood was collected into tubes containing sodium citrate as an anticoagulant. The blood was centrifuged to obtain plasma and was analyzed for activated partial thromboplastin time (APTT), prothombin time (PT) and fibrinogen. For APTT and PT determination, samples were obtained pre-infusion, end of infusion and 4 h post-infusion on the first and last dosing days. Fibrinogen was assayed only pre-infusion on the first day and 24 hours after the last dose.

For the assessment of complement parameters, approximately 0.75 mL of blood was collected into EDTA-containing tubes to obtain plasma for complement split product (Bb) analysis. An additional 0.75 mL was collected into a tube without anticoagulant and serum collected by centrifugation for the analysis of CH₅₀. Plasma and serum collected as described above were frozen on dry ice and stored at −70° C. until analysis.

Urinalysis

Urine samples were collected from the bladder at necropsy and analyzed for color/character, pH, specific gravity, leukocyte esterase, nitrite, urobilinogen, protein, glucose, ketones, bilirubin, occult blood and microscopic contents.

Toxicokinetic Samples and INX-3280 Assay

Approximately 0.75 mL of blood was collected into EDTA-containing tubes at the end of infusion, 4 h post-infusion and 24 h post-infusion after the first and last doses as well as prior to the first infusion. Plasma was obtained by centrifugation and the samples were frozen on dry ice and stored at −70° C. prior to analysis. INX-3280, as well as the N-1, N-2 and N-3 chain-shortened (3′-end) variations, were quantified in the thawed plasma samples using a validated HPLC method. Reported pharmacokinetic parameters were obtained using WinNonlin pharmacokinetic sofware (Version 3.0; Pharsight, Inc., CA) using a non-compartmental analysis. Very similar parameters were obtained using a noncompartmental pharmacokinetic analysis program (Version 2.5; Dr. J. Venitz, Department of Pharmacy and Pharmaceutics, Medical College of Virginia, VCU).

Necropsy and Histopathology

The animals were terminated by exsanguination while under deep anesthesia. A final body weight was obtained and a complete gross necropsy conducted. Various organs were weighed and then embedded in paraffin, sectioned, stained with hematoxylin and eosin, and examined by light microscopy.

In vivo Toxicology Results

No animals died during the course of the study and there were no abnormal clinical signs indicative of an adverse effect arising from the administration of INX-3280. Body weight data indicated no treatment-related changes during the course of the study. Data for mean blood pressure and heart rate indicate no changes that were associated with the administration of INX-3280.

Hematology parameters were determined on the first and last dosing days. Small changes were observed in neutrophil, monocyte and lymphocyte counts that were generally maximal at the end of infusion. However, it is likely that these changes arose from the stress associated with the experimental procedures, as similar changes were observed in both INX-3280-treated and control groups. In addition, the indicators of circulating red blood cell mass (RBC counts, hemoglobin concentration and hematocrit) slowly decreased during the study due to blood sampling. No treatment-related alterations were observed for band cells, eosinophils, basophils or reticulocytes. Overall, there were no alterations in the hematology parameters that were associated with the administration of INX-3280.

Serum chemistry parameters indicated no significant treatment-associated changes. Several high values for serum enzymes (LDH, AST and ALT) observed in some animals prior to the first dose were attributed to minor tissue damage occurring during catheter insertion and animal restraint. Data for serum concentrations of sodium, potassium, chloride, CO₂, calcium, phosphorous, cholesterol and triglycerides indicated no treatment-associated alterations in these components. In addition, the urinalysis indicated no changes that were associated with the administration of INX-3280.

With respect to coagulation parameters, minor increases (approximately 30%) in APTT values were observed at the end of infusion of INX-3280 in animals that received 15 mg/kg, both on Day 1 and Day 12. This prolongation of APTT was reversed in the 15 mg/kg group by 4 hours after the infusion. Similar trends were observed in PT values, but were less apparent than the APTT alterations. No change in APTT or PT was evident in animals administered INX-3280 at 3.0 mg/kg. No treatment-associated alterations in fibrinogen concentrations were observed.

To assess the effects of INX-3280 on complement activation, both the alternative complement pathway split product, Bb, and the total hemolytic complement potential, CH₅₀, were measured. Only minor increases in Bb were observed during INX-3280 infusion. These changes were attributed to the stress of the experimental procedures because the magnitude of the increase was similar across groups (including the control group) and was unrelated to the dose of INX-3280 administered. Similar small increases in Bb are typically seen under these experimental conditions, whereas much larger increases occur when complement is activated by phosphorothioate oligonucleotides. No significant changes in CH₅₀ were observed during the course of this study. Overall, there were no alterations in the complement activation parameters that were associated with the administration of INX-3280.

Gross necropsies were performed on all animals on Day 15 of the study. No macroscopic changes were observed that were related to treatment with INX-3280. No changes in organ weights were observed that were associated with the administration of INX-3280. Treatment-related changes were observed in the kidneys, liver, and mesenteric and mandibular lymph nodes. The typical observation was the presence of basophilic granular material in the cytoplasm of epithelial cells of the proximal tubules of the kidneys and of reticuloendothelial cells in the lymph nodes and liver. Note that all lesions were graded as minimal in animals treated at 3.0 and at 15.0 mg/kg.

Pharmacokinetics

Pharmacokinetic parameters were determined after the administration of INX-3280 at either 3.0 or 15 mg/kg. After the first and sixth administrations of INX-3280 at 15 mg/kg, the plasma concentrations, when compared at each time point, were statistically identical. These results suggest that the clearance of INX-3280 from the blood of monkeys was not altered by the repeated administration schedule for this oligonucleotide. This is supported by the observation that the estimated clearance halflife (T_{½}) of INX-3280 after the first administration (2.85 h) was nearly identical to that after the sixth administration (2.89 h). A similar relationship between the first and sixth administrations was observed in animals treated at 3.0 mg/kg of INX-3280. At this dose, it was not possible to estimate the T_{½} in this group of animals due to the 24 h time point having INX-3280 concentrations below the limit of quantitation of the validated HPLC assay.

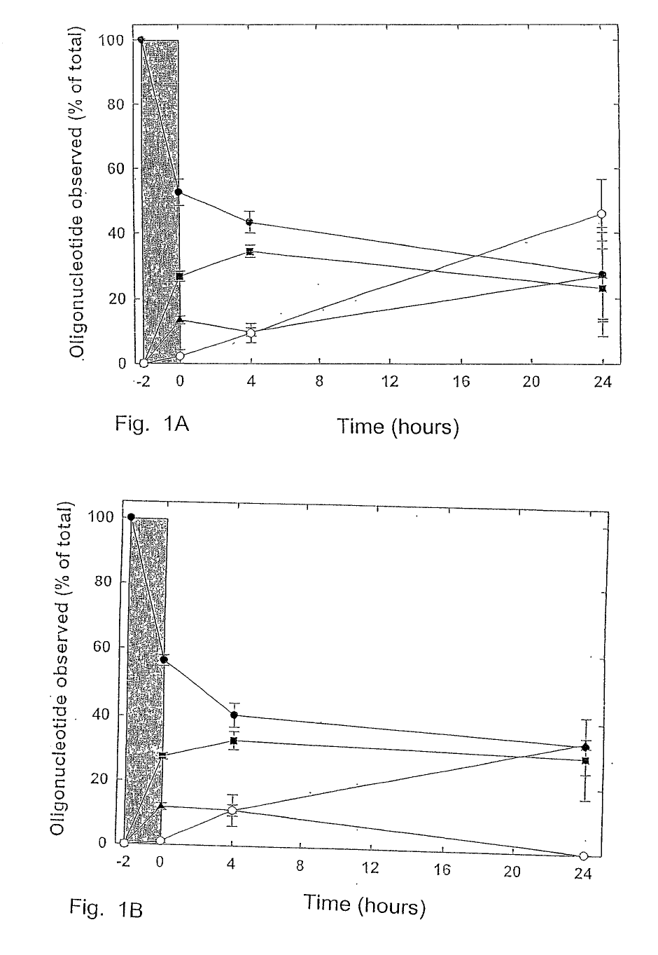

Phosphorothioate oligonucleotides are metabolized in vivo, primarily via exonuclease activity at the 3-prime terminus (Geary et al., 1997). Consequently, the validated HPLC method was designed to quantify the full-length oligonucleotide, INX-3280, as well as the principle metabolites having one (N-1), two (N-2) or three (N-3) bases removed. FIG. 1 shows that intact INX-3280 comprised 52.5 and 56.5% of the total oligonucleotide present in the plasma at the completion of infusion on the first and sixth administrations, respectively, at 15 mg/kg. The proportion of oligonucleotide in the plasma of cynomolgous monkeys after the first (day 1; Panel A) or sixth (day 12; Panel B) i.v. administrations of INX-3280 at 15 mg/kg. Proportions are reported for the full-length, intact, INX-3280 (●), the N-1 (▪) and the N-2 (▴) sequences as well as for the summed unknown peaks (◯). Values represent the mean (±standard deviations) from four animals/group. The grey bar represents the 2 hour infusion period.

As shown, at the completion of infusion, the intact INX-3280, plus the N-1 and N-2 metabolites, comprised greater than 93% of the total oligonucleotide in the plasma. At 4 h after the end of infusion, INX-3280 plus the N-1 and N-2 metabolites remained the principle components, representing 88 and 84%, respectively, of total oligonucleotide present in the plasma. At 24 h after infusion, INX-3280 represented 27.4 and 33.4% of the total oligonucleotide after the first and sixth administrations, respectively. After the first administration, the remaining oligonucleotides were comprised predominantly of the N-1 (23.1%) and N-2 (27.0%) as well as unknown peaks (46.0%). In contrast, after the sixth administration, the remaining oligonucleotides were comprised primarily of the N-1 (29.4%) and N-2 (34.2%) metabolites, with no detectable unknown peaks. This latter observation is the only distinction between the pharmacokinetics of INX-3280 after the first and sixth doses. The proportions of the N-3 metabolite of INX-3280 were low (<8.8%) at all time points investigated.

A key finding of this invention may be drawn from this Example. Here are reported the results of a 2-week toxicity study in cynomolgous monkeys in which INX-3280 was given 3 times a week as 2-hour infusions. The dose schedule was analogous to a proposed clinical regimen for INX-3280. In addition to characterizing complement activation under clinically relevant dosing conditions, this study also investigated INX-3280 inhibition of the intrinsic coagulation pathway, reflected by prolongation of APTT. Both complement activation and inhibition of coagulation are blood-level-related class effects of phosphorothioate oligonucleotides (Levin et al., 1998). A novel aspect of this study was the implementation of a heating step in the preparation of dosing solutions that converted the portion of the oligonucleotide in solution that normally assumes a complex tertiary structure (quadruplex) to its simple monomeric form.

The results of this study contrasted sharply with those of previous studies of phosphorothioate oligonucleotides in monkeys. At dose levels up to 15 mg/kg/infusion, no animals died, and there were no clinical signs or changes in appetite, body weight, blood pressure, heart rate, serum chemistry parameters, hematology indices, complement split product Bb and CH50 values, urinalysis parameters, organ weights and gross pathology that were related to INX-3280. The only effect on coagulation parameters was a modest increase in APTT at the end of infusion in the group that received the 15 mg/kg doses (but not in the lower dose group treated with 3 mg/kg/infusion). The prolongation of APTT in the 15 mg/kg group was only 30 percent or less above the pre-study mean, which is not considered clinically significant. Furthermore, this alteration was reversed within hours after the end of infusion in parallel with the clearance of the oligonucleotide from circulation. The magnitude of the increase in APTT at 15 mg/kg is less than that observed with other phosphorothioate oligonucleotides under similar dosing conditions (Henry et al., 1997b; Henry et al., 1997c; Monteith et al. 1998).

Similarly, the absence of complement activation by INX-3280 given at the 15-mg/kg dose level is unusual when compared to other compounds in this class (Henry et al. 1997a; Henry et al., 1997b; Levin et al. 1998; Monteith et al., 1998). Activation of the alternative pathway by phosphorothioate oligonucleotides is a well recognized class effect in monkeys that can lead to life-threatening hemodynamic disturbances (Galbraith et al., 1994; Henry et al., 1997a; Henry et al., 1997b; Monteith et al., 1998). Fortunately, this effect is blood-level dependent and can be ameliorated by diminishing the rate of intravenous infusion (i.e., by increasing the duration of infusion to deliver the desired dose). It is important to note that the plasma concentration of full-length INX-3280 at the end of the 15-mg/kg infusions (101-120 (g/mL; Table 9) was well above the threshold concentration for complement activation of 40-50 (g/mL that has been reported for other phosphorothioate oligonucleotides (Henry et al., 1997a; Henry et al., 1997b; Monteith et al., 1998; Levin et al., 1998). These results are consistent with the in vitro observation reported in other Examples attached hereto that monomeric INX-3280 at 100 (g/mL has reduced complement consumption compared to both the unmonomerized INX-3280 and INX 2302 administered at the same concentrations.

This difference, compared to other phosphorothioate oligonucleotides, is apparently attributable to the use of monomeric INX-3280 in the study, which may reduce the potential for interactions with blood proteins. This interpretation is directly supported by the in vitro comparison of the complement and coagulation activities of unmonomerized and monomeric INX-3280 and with INX 2302. In these in vitro assays, the monomeric INX-3280 was significantly less able to activate complement and inhibit coagulation when compared to the unmonomerized INX-3280, but was also improved compared to INX 2302. These observations parallel the in vivo determinations of complement activation and coagulation by monomeric INX-3280. For example, in cynomolgus monkeys administered doses of 10 mg/kg of a phosphorothioate oligonucleotide against c-raf kinase, peak plasma concentrations of oligonucleotide of 35 (g/ml were associated with complement activation (Bb>1 (g/mL) and 50-70% increases in APTT (Monteith et al., 1998). Similarly, cynomolgus monkeys given INX 2302 had activation of the complement cascade associated with a plasma concentration of approximately 50 (g/mL (Henry et al., 1997a) and >50% increases in APTT at plasma oligonucleotide concentrations of approximately 80 (g/mL (Henry et al., 1997c). In marked contrast, peak INX-3280 concentrations in the plasma of 101.5 to 119.6 (g/mL in monkeys treated at 15 mg/kg (Table 9) were associated with no significant increases in Bb values ((1 (g/mL; Table 7) and only minor (30%) increases in APTT values (Table 6).

The only finding from the microscopic examination of tissues obtained from the monkeys after the 2 weeks of dosing with INX-3280 was the presence of basophilic granular material with tubular epithelial cells in the kidneys and within reticuloendothelial cells of the liver (Kupffer cells) and lymph nodes (resident macrophages). These alterations were present in both INX-3280-treated groups, but were graded as minimal in all animals. Based on work performed with other oligonucleotides, the granular material is believed to be the oligonucleotide itself or related substances (possibly protein-bound metabolites) that accumulate in these cells (Levin et al., 1998; Monteith et al., 1998). This is a benign alteration that is not associated with any degenerative lesions or alterations in organ function, and simply reflects deposition and ongoing clearance of the oligonucleotide. As such, it is not regarded as a toxicologically significant finding. Hence, when monomerized INX-3280 was given by 2-hour intravenous infusion 3 times per week for 2 weeks, there were no significant adverse effects at a dose level of 15 mg/kg/infusion.

The disposition of phosphorothioate oligonucleotides has been well characterized, and it is widely accepted that all compounds in this class behave very similarly, and that the kinetics and tissue distribution are similar across species (Cossum et al., 1993; Sands et al., 1994; Bennett et al. 1996, Geary et al., 1997). The sampling times used here were not intended to duplicate these earlier studies. Nonetheless, the clearance half-lives reported in Table 8 are consistent with the published reports (reviewed by Geary et al., 1997) that oligonucleotides are cleared fairly rapidly from the blood compartment. Importantly, the rate of clearance of INX-3280 from the blood compartment was identical on the sixth administration compared to the first. That is, the clearance rate was not affected by prior INX-3280 administrations, in spite of the slow metabolism (typical half-lives of 1-2 days) of oligonucleotides in the major organs of distribution (kidneys and liver) (Geary et al., 1997).

It is now well established that oligonucleotides are extensively metabolized in the blood compartment, mainly by exonuclease-mediated activity, yielding “chain-shortened” products. The data presented in FIG. 1 is consistent with the exonuclease-mediated metabolism of INX-3280 to its N-1 and N-2 metabolic products. Since low levels of the N-3 product were observed at all sampled times, the appearance at later times after INX-3280 administration of a variety of unknown catabolic products may indicate a role for endonuclease activity in the metabolism of INX-3280.

Taken together, these results demonstrate that monomeric INX-3280 has an excellent safety profile. INX-3280 given in this 2-week study in monkeys was administered at dose levels up to 15 mg/kg in a schedule identical to that intended for the initial clinical trials. Those toxicities stemming from interaction with blood proteins, complement activation and inhibition of coagulation, have been a primary concern for development of other oligonucleotides. However, it appears that the process of “monomerizing” the INX-3280 substantially reduces the potential for eliciting these toxicities and substantially increases the margin of safety for INX-3280, relative to other compounds in this class.

### EXAMPLE 3

For in vitro studies, the phosphorothioate oligonucleotide INX 2302 was manufactured according to current Good Manufacturing Practices using methods described previously (Fearon et al., 1997). INX 2302 was dissolved in water and stored at 2-8° C. INX 2302 is a 20-mer sequence against ICAM-1 mRNA having the sequence:

INX 2302: 5′-GCC CAA GCT GGC ATC CGT CA-3′ Seq. ID. No. 2

In vitro Complement and Coagulation Assays

The determination of total complement activity was assessed in human serum using an enzyme immunoassay kit (DiaSorin, Stillwater Minn.) as described by the manufacturer. Duplicate oligonucleotide samples, suspended in veronal-buffered saline, were diluted 1:9 with normal human serum, then tested at final concentrations between 0.1 and 100 μg/mL.

Coagulation activity was determined by measuring Activated Partial Thromboplastin Time (APTT) using the Dade Actin FS ATPP reagent (Dade Behring, Deerfield, Ill.). Assays were conducted as described by the manufacturer using normal human plasma anticoagulated with 3.8% sodium citrate. Duplicate oligonucleotide samples, suspended in veronal-buffered saline, were diluted 1:9 with normal human plasma, then tested at final concentrations between 0.1 and 100 μg/mL.

In vivo data discussed in Example 2 strongly suggest that monomerization of INX-3280 reduces it ability to elicit complement activation and inhibit coagulation. However, a direct comparison of the monomer and the quadruplex was not possible as monkeys were not administered with INX-3280 in the quadruplex form because of its toxicty. Consequently, the complement and coagulation activities of the monomeric and quadruplex forms of INX-3280 were compared using in vitro assays.

In whole blood exposed to oligonucleotides prior to coagulation, both ISIS 2302 and the monomerized (<2% quadruplex; Table 1) INX-3280 were relatively non-activating. Specifically, in whole blood incubated with oligonucleotide at 50 mg/mL, the complement activity remaining after 30 minutes was 128 and 152 CAE units, respectively. In contrast, the unmonomerized INX-3280 (54% quadruplex; Table 1) consumed most of the available complement, with 34 CAE units remaining at the end of the incubation. The differences between these oligonucleotides were more pronounced after incubation at 100 mg/mL. Complement activity remaining after this incubation was 101, 19 and 36 CAE units for monomerized INX-3280, unmonomerized INX-3280 and ISIS 2302, respectively. Similar results were observed when normal human serum was exposed to the oligonucleotides.

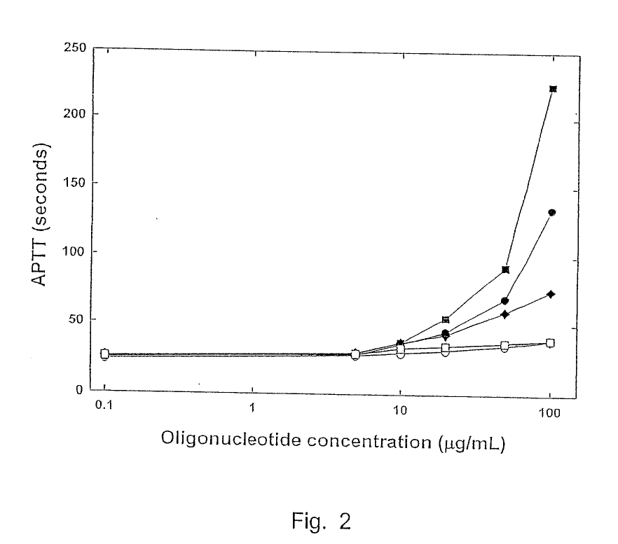

Effects of oligonucleotides on coagulation were assessed by determination of the Activated Partial Thromboplastin Time (APTT), and the results are summarized in FIG. 2. Activated Partial Thromboplastin Time (APTT; seconds) was determined in serum derived from normal human blood that was treated with various concentrations of oligonucleotides Blood was treated with a control oligonucleotide, ISIS 2302 (♦), or with either Batch #1 of INX-3280 in unmonomerized (●) or monomeric (◯) forms or with Batch #2 of INX-3280 in unmonomerized (▪) or monomeric (□) forms. Data represents the mean values from duplicate tests.

Normal APTT values were in the range from 24.4 to 28.6 seconds. When added at concentrations up to 100 mg/niL, ISIS 2302 increased the APTT to values of 74.4 seconds (FIG. 2). The unmonomerized INX-3280 was significantly more inhibitory of blood coagulation (FIG. 2). Specifically, the APTT values observed at 100 mg/mL of INX-3280 were in the range between 134 seconds (Batch #1) and 225.4 seconds (Batch #2; Table 1). Monomerization of INX-3280 effectively ameliorated the inhibition of blood coagulation (FIG. 2). Monomerization of INX-3280 (Table 1) was reflected as a significant reduction in the APTT values at 100 mg/ml of oligonucleotide from 134-225 seconds to 39.1 seconds (Batch #1) to 39.7 seconds (Batch #2; FIG. 2).

### EXAMPLE 4

Experiment 1: Influence of Initial INX-3280 Concentration on Monomer:Quadruplex Ratio After Lyophilization from WFI.

Monomer:quadruplex ratio for INX-3280 can be influenced by oligonucleotide concentration in solution and we therefore examined the effect of this parameter on the ratio observed in product lyophilized from WFI (Water for Injection). Starting with BDS (Bulk Drug Substance, i.e, a batch of oligo in solution) that was rendered essentially 100% monomer by heating, we prepared dilutions of INX-3280 from 1.5 mg/ml to 12.0 mg/ml in WFI and then lyophilized these samples using the cycle shown below. Following drying, all samples exhibited a similar appearance with a uniform amorphous cake slightly contracted from the vial walls.

Lyophilization Cycle

| Step | Shelf Temp. | Vacuum | Time (Duration) | |
|---|---|---|---|---|
| 1 | −50° C. | Atmos. | 2 | Hr |
| 2 | −50° C. | 100 microns | 1.5 | Hr |
| 3 | −25° C. | 100 microns | 1 | Hr |
| 4 | 0° C. | 100 microns | 8 | Hrs |
| 5 | +40° C. | 100 microns | 8 | Hrs |
| 6 | +25° C. | 100 microns | 2 | Hrs |

The results are summarized in Table 2. As shown, INX-3280 BDS exhibits a monomer:quadruplex ratio of approximately 51:49. Heating at 65° C. for 30 minutes converts essentially all quadruplex to monomer. Lyophilization of this monomer solution, however, results in significant formation of quadruplex with the absolute ratio being slightly influenced by the initial oligonucleotide concentration. At an initial INX-3280 concentration of 1.5 mg/ml the monomer percentage after lyophilization and rehydration is approximately 50% but this falls to approximately 45% when the initial oligomer concentration is 12.0 mg/ml. It should be noted that the lyophilized samples are all rehydrated to the same final concentration, 2.5 mg/ml, and so these differences do not reflect a concentration-dependent, post-rehydration formation of quadruplex. In view of the lack of a substantial effect of initial oligomer concentration on monomer:quadruplex ratio, subsequent studies mostly examined samples containing 6.7 mg/ml INX-3280 which is the concentration currently used in preparation of clinical product.

TABLE 2

| | Quadruplex | |
|---|---|---|
| Sample | (% Area) | Monomer (% Area) |
| Initial BDS | 48.83 | 51.17 |
| INX-3280 post 65° C. incubation | 0.76 | 99.24 |
| Lyophilized, 1.5 mg/ml front | 49.04 | 50.96 |
| Lyophilized, 1.5 mg/ml middle | 49.30 | 50.70 |
| Lyophilized, 1.5 mg/ml rear | 53.21 | 46.79 |
| Lyophilized, 3.0 mg/ml front | 53.68 | 46.32 |
| Lyophilized, 3.0 mg/ml middle | 51.22 | 48.78 |
| Lyophilized, 3.0 mg/ml rear | 51.27 | 48.43 |
| Lyophilized, 6.7 mg/ml front | 54.87 | 45.13 |
| Lyophilized, 6.7 mg/ml middle | 51.76 | 48.24 |
| Lyophilized, 6.7 mg/ml rear | 55.68 | 44.32 |
| Lyophilized, 12 mg/ml front | 55.36 | 44.64 |
| Lyophilized, 12 mg/ml middle | 53.79 | 46.21 |
| Lyophilized, 12 mg/ml rear | 54.98 | 45.02 |

Experiment 2: Influence of Freezing Time on INX-3280 Monomer:Quadruplex Ratio After Lyophilization from WFI

Monomer solution of INX-3280 will form a substantial proportion of quadruplex on freezing alone. Further, the rate of cooling, or total freezing time, may influence the extent of quadruplex formation. We therefore employed a modified lyophilization cycle (see below) that extended the total product freezing time to determine if this parameter had a significant impact on the monomer:quadruplex ratio of INX-3280. Again the oligonucleotide was lyophilized from WFI with two concentrations being tested, 1.5 mg/ml and 6.7 mg/ml.

Lyophilization Cycle

| Step | Shelf Temp. | Vacuum | Time (Duration) | |
|---|---|---|---|---|
| 1 | −50° C. | Atmos. | 4 | Hr |
| 2 | −50° C. | 100 microns | 2 | Hr |
| 3 | −25° C. | 100 microns | 1 | Hr |
| 4 | 0° C. | 100 microns | 8 | Hrs |
| 5 | +40° C. | 100 microns | 8 | Hrs |
| 6 | +25° C. | 100 microns | 2 | Hrs |

TABLE 3

| | Quadruplex | |
|---|---|---|
| Sample | (% Area) | Monomer (% Area) |
| Initial BDS | 48.83 | 51.17 |
| INX-3280 post 65° C. incubation | 0.76 | 99.24 |
| Lyophilized, 1.5 mg/ml front a | 54.57 | 45.43 |
| Lyophilized, 1.5 mg/ml front b | 51.12 | 48.88 |
| Lyophilized, 1.5 mg/ml middle a | 52.17 | 47.83 |
| Lyophilized, 1.5 mg/ml middle b | 54.40 | 45.60 |
| Lyophilized, 1.5 mg/ml rear a | 55.10 | 44.90 |
| Lyophilized, 1.5 mg/ml rear b | 50.74 | 49.26 |
| Lyophilized, 6.7 mg/ml front a | 55.67 | 44.33 |
| Lyophilized, 6.7 mg/ml front b | 54.84 | 45.16 |
| Lyophilized, 6.7 mg/ml middle a | 55.51 | 44.49 |
| Lyophilized, 6.7 mg/ml middle b | 54.40 | 45.60 |
| Lyophilized, 6.7 mg/ml rear a | 55.74 | 44.26 |
| Lyophilized, 6.7 mg/ml rear b | 53.65 | 46.35 |

The data shown in Table 3 indicate that increasing the freezing time from the two hours used in the first lyophilization cycle to four hours does not significantly influence the monomer:quadruplex ratio of the rehydrated product. At an initial INX-3280 concentration of 1.5 mg/ml there is possibly a small increase in the percentage of quadruplex (c.f. Table 2) but at 6.7 mg/ml no differences are apparent. This result indicates that holding the vialed INX-3280 solution on the lyophilizer shelf at −50° C. for two hours is sufficient to ensure complete freezing.

### EXAMPLE 5

Cryoprotectants such as sucrose and mannitol are used to protect proteins from denaturation during lyophilization. Part of this protective effect likely reflects their ability to inhibit aggregation. Such cryoprotectants could therefore be of potential utility in maintaining INX-3280 in a monomeric form during lyophilization. We therefore lyophilized INX-3280 from solutions containing either sucrose (300 mM) or mannitol (300 mM) at an oligonucleotide concentration of 6.7 mg/ml and examined the monomer:quadruplex ratio of the rehydrated sample. Use of these excipients required that the lyophilization cycle be significantly modified from that used during drying of the compound from WFI (see below). The resulting dry samples were uniform amorphous cakes with little or no shrinkage from the walls of the vial.

Lyophilization Cycle

| Step | Shelf Temp. | Vacuum | Time (Duration) | |
|---|---|---|---|---|
| 1 | −50° C. | Atmos. | 3 | Hr |
| 2 | −30° C. | 100 microns | 30 | Hr |
| 3 | 0° C. | 100 microns | 2 | Hrs |
| 4 | +25° C. | 100 microns | 4 | Hrs |

As shown in Table 4, both sucrose and mannitol inhibit quadruplex formation during lyophilization of INX-3280. Samples dried in the presence of mannitol exhibit approximately 5% quadruplex formation, while product dried in the presence of sucrose exhibits less than 2% quadruplex. This result confirms that sucrose and mannitol are effective cryoprotectants and can inhibit quadruplex formation during lyophilizaton.

TABLE 4

| | Quadruplex | Monomer |
|---|---|---|
| Sample | (% Area) | (% Area) |
| Initial BDS | 48.83 | 51.17 |
| INX-3280 post 65° C. incubation | 0.19 | 99.81 |
| Sucrose 300 mM, 6.7 mg/ml front a | 0.02 | 99.98 |
| Sucrose 300 mM, 6.7 mg/ml front b | 0.02 | 99.98 |
| Sucrose 300 mM, 6.7 mg/ml middle a | 0.03 | 99.97 |
| Sucrose 300 mM, 6.7 mg/ml middle b | 0.03 | 99.97 |
| Sucrose 300 mM, 6.7 mg/ml rear a | 1.22 | 98.78 |
| Sucrose 300 mM, 6.7 mg/ml rear b | 0.02 | 99.98 |
| Mannitol 300 mM, 6.7 mg/ml front a | 4.77 | 95.23 |
| Mannitol 300 mM, 6.7 mg/ml front b | 3.66 | 96.34 |
| Mannitol 300 mM, 6.7 mg/ml middle a | 4.83 | 95.17 |
| Mannitol 300 mM, 6.7 mg/ml middle b | 4.86 | 95.14 |
| Mannitol 300 mM, 6.7 mg/ml rear a | 4.82 | 95.18 |
| Mannitol 300 mM, 6.7 mg/ml rear b | 4.84 | 95.16 |

### EXAMPLE 6

Following the demonstration that 300 mM sucrose can fully protect INX-3280 against quadruplex formation during drying, we examined the concentration dependent nature of this protection. Oligonucleotide was lyophilized at 6.7 mg/ml from 0, 10, 50, 100, 300 and 500 mM sucrose using the cycle shown in Example 5. This experiment also allows a comparison between the new lyophilization cycle and that used in Experiments 1 and 2 of Example 4 with regard to quadruplex formation for INX-3280 dried from WFI (i.e. 0 mM sucrose).

As can be seen from Table 5, there is a concentration-dependent protection by sucrose with significant inhibition of quadruplex formation even at 10 mM: Interestingly, this represents about 3.4 mg/ml sucrose i.e. approximately half the weight of INX-3280. Essentially complete protection against quadruplex formation is achieved at and above 50 mM sucrose. This behavior is consistent with sucrose acting as a non-eutectic cryoprotectant and being concentrated with the oligonucleotide during ice crystal formation. It is of interest to note that the quadruplex:monomer ratio of samples lyophilized in WFI (0 mM sucrose) is similar to the ratios observed in Tables 2 and 3 notwithstanding the considerable differences between the lyophilization cycles used in these three experiments.

TABLE 5

| Sample | Quadruplex (% Area) | Monomer (% Area) |
|---|---|---|
| 0 mM Sucrose front a | 56.45 | 43.50¹ |
| 0 mM Sucrose front b | 54.05 | 45.95 |
| 0 mM Sucrose middle a | 54.53 | 45.47 |
| 0 mM Sucrose middle b | 53.93 | 46.07 |
| 0 mM Sucrose rear a | 56.18 | 43.82 |
| 0 mM Sucrose rear b | 54.37 | 45.63 |
| 10 mM Sucrose front a | 25.42 | 74.58 |
| 10 mM Sucrose middle a | 23.29 | 76.71 |
| 10 mM Sucrose middle b | 24.37 | 75.63 |
| 10 mM Sucrose rear a | 23.99 | 76.01 |
| 10 mM Sucrose rear b | 24.42 | 75.58 |
| 50 mM Sucrose front a | 0.19 | 99.81 |
| 50 mM Sucrose front b | 0.18 | 99.82 |
| 50 mM Sucrose middle a | 0.19 | 99.81 |
| 50 mM Sucrose middle b | 0.27 | 99.73 |
| 50 mM Sucrose rear a | 0.13 | 99.87 |
| 50 mM Sucrose rear b | 0.10 | 99.90 |
| 100 mM Sucrose front a | Not detected | 100.0 |
| 100 mM Sucrose front b | Not detected | 100.0 |
| 100 mM Sucrose middle a | Not detected | 100.0 |
| 100 mM Sucrose middle b | Not detected | 100.0 |
| 300 mM Sucrose front a | Not detected | 100.0 |
| 300 mM Sucrose rear a | Not detected | 100.0 |
| 500 mM Sucrose front a | Not detected | 100.0 |
| 500 mM Sucrose front b | Not detected | 100.0 |

¹Note.
Minor contaminant peak seen hence percentages do not total to 100% for this sample.

### REFERENCES

The following references, all of which are incorporated herein by reference, are discussed in the foregoing description of the invention:

Ahn, C.-H. and DeGeorge, J. J. (1998). Preclinical development of antisense oligonucleotide therapeutics for cancer: Regulatory aspects. In Clinical Trials of Genetic Therapy with Antisense DNA and DNA Vectors. E. Wickstrom ed., (Marcel Dekker, Inc., New York), pp. 39-52.

Basu, S., and Wickstrom, E. (1997). Temperature and salt dependence of higher order structure formation by antisense c-myc and c-myb phosphorothioate oligodeoxyribonucleotides containing tetraguanylate tracts. Nucleic Acids Research 25, 1327-1332.

Bennett, C. F., Zuckerman, J. E., Kombrust, D., Sasmor, H., Leeds, J. M., and Crooke, S. T. (1996). Pharmacokinetics in mice of a [³H]-labeled phosphorothioate oligonucleotide formulated in the presence and absence of a cationic lipid. Journal of Controlled Released 41, 121-130.

Black, L. E., Farrelly, J. G., Cavagnaro, J. A., Ahn, C.-H., DeGeorge, J. J., Taylor, A. S., DeFelice, A. F., and Jordan, A. (1994). Regulatory considerations for oligonucleotide drugs: Updated recommendations for pharmacology and toxicology studies. Antisense Research and Development 4, 299-301.

Cheng A. J., and van Dyke M. W. (1997). Oligodeoxyribonucleotides length and sequence effects on intramolecular and intermolecular G-quartet formation. Gene 197, 253-260.

Cossum, P. A., Sasmor, H., Dellinger, D., Troung, L., Cummins, L., Owens, S. R., Markham, P. M., Shea, J. P., and Crooke, S. (1993). Disposition of the ¹⁴C-labeled phosphorothioate oligonucleotide ISIS 2105 after intravenous administration to rats. Journal of Pharmacology and Experimental Therapeutics 267, 1181-1190.

Galbraith, W. M., Hobson, W. C., Giclas, P. C., Schecter, P. J., and Agrawal, S. (1994). Complement activation and hemodynamic changes following intravenous administration of phosphorothioate oligonucleotides in the monkey. Antisense Research and Development 4, 201-206.

Geary, R., Leeds, J., Henry, S. P., Monteith, D. K., and Levin, A. A. (1997). Antisense oligonucleotide inhibitors for the treatment of cancer. 1. Pharmacokinetic properties of phosphorothioate oligonucleotides. Anticancer Drug Design 12, 383-393.

Heikkila, R., Schwab, G., Wickstrom, E., Loke, S. L., Pluznik, D. H., Watt, R., and Neckers, L. M. (1987). A c-myc antisense oligodeoxynucleotide inhibits entry into S phase but not progress from G₀ to G₁. Nature 328, 445-449.

Henry, S. P., Giclas, P. C., Leeds, J., Pangburn, M., Auletta, C., Levin, A. A., and Kombrust D. J. (1997a). Activation of the alternative pathway of complement by a phosphorthioate oligonucleotide: Potential mechanism of action. Journal of Pharmacology and Experimental Therapeutics 281, 810-816.

Henry, S. P., Monteith, D., Kombrust, D. J., and Levin, A. A. (1 997b). Effects of intravenous infusion of phosphorothioate oligonucleotides on coagulation, complement activation and hemodynamics. Nucleosides and Nucleotides 16, 1673-1676.

Henry, S. P., Novotny, W., Leeds, J., Auletta, C., and Kornbrust, D. J. (1997c). Inhibition of coagulation by a phosphorothio ate oligonucleotide. Anti sense and Nucleic Acid Drug Development 7, 503-510.

Levin, A. A., Monteith, D. K., Leeds, J. M., Nicklin, P. L., Geary, R. S., Butler, M., Templin, M. V., and Henry, S. P. (1998). Toxicity of oligonucleotide therapeutic agents. In: Handbook of Experimental Pharmacology. G. V. R. Born et al., ed. (Springer-Verlag, Berlin), pp. 169-215.

Macaya, R. F., Waldron, J. A., Beutel, B. A., Gao, H., Joesten, M. E., Yang, M., Patel, R., Bertelsen, A. H., and Cook, A. F. (1995). Structural and functional characterization of potent antithrombotic oligonucleotides possessing both quadruplex and duplex motifs. Biochemistry 34, 4478-4492.

Monteith, D. K., Geary, R. S., Leeds, J. M., Johnston, J., Monia, B. P., and Levin, A. A. (1998). Preclinical evaluation of the effects of a novel antisense compound targeting c-raf kinase in mice and monkeys. Toxicological Sciences 46, 365-375.

Pearson, A. M., Rich, A., and Krieger, M. (1993). Polynucleotide binding to macrophage scavenger receptors depends on the formation of base-quartet-stabilized four-stranded helices. Journal of Biological Chemistry 268, 3546-3554.

Sands, H., Gorey-Feret, L. J., Cocuzza, A. J., Hobbs, F. W., Chidester, D., and Trainor, G. L. (1994). Biodistribution and metabolism of internally ³H-labeled oligonucleotides. I. Comparison of a phosphodiester and phosphorothioate. Molecular Pharmacology 45, 932-943.

Suzuki, K., Doi, T., Irnanishi, T., Kodama, T., and Tanaka, T. (1999). Oligonucleotide aggregates bind to the macrophage scavenger receptor. European Journal of Biochemistry 260, 855-860.

What is claimed is:

1. A method for administration of therapeutic oligonucleotides which tend to form multimeric aggregates comprising the steps of:
(a) treating a composition comprising the therapeutic oligonucleotide in multimeric aggregate form to convert substantially all of the therapeutic oligonucleotide to a monomeric form or to substantially prevent the formation of aggregates; and
(b) administering the composition in which substantially all of the therapeutic oligonucleotide is in monomeric form to a mammal in need of therapy provided by the oligonucleotide.

2. The method of claim 1, wherein the therapeutic oligonucleotide is a 5 to 50 mer.

3. The method of claim 2, wherein the composition comprising the therapeutic oligonucleotide is treated by heating the composition.

4. The method of claim 3, wherein the composition is heated to a temperature of from 60 to 90° C. for a period of 3 to 60 minutes.

5. The method of claim 1, wherein the therapeutic oligonucleotide comprises a 4G motif.

6. The method of claim 1, wherein the composition comprising the therapeutic oligonucleotide is treated by heating the composition.

7. The method of claim 6, wherein the composition comprising the therapeutic oligonucleotide is treated by heating less than 24 hours prior to administration.

8. The method of claim 6, wherein the composition is heated to a temperature of from 60 to 90° C. for a period of 3 to 60 minutes.

9. The method of claim 8, wherein the composition comprising the therapeutic oligonucleotide is treated by heating less than 24 hours prior to administration.

10. The method of claim 1 wherein, wherein the therapeutic composition is treated by addition of a chemical additive effective to substantially prevent aggregation.

11. The method of claim 10, wherein the chemical additive is mannitol or sucrose.

12. The method of claim 10, wherein the therapeutic oligonucleotide is a 5 to 50 mer.

13. The method claim 10, further comprising the steps of:
lyophilizing the treated composition to form a dried product in which substantially all of the oligonucleotide is present in monomeric form; and
reconstituting the lyophilized product prior to administration to form a reconstituted composition in which substantially all of the oligonucleotide is present in monomeric form, wherein the reconstituted product is used in the administration step (b).

14. The method of claim 10, wherein the oligonucleotides are phophorothioate oligonucleotides.

15. The method claim 1, further comprising the steps of:
lyophilizing the treated composition to form a dried product in which substantially all of the oligonucleotide is present in monomeric form; and
reconstituting the lyophilized product prior to administration to form a reconstituted composition in which substantially all of the oligonucleotide is present in monomeric form, wherein the reconstituted product is used in the administration step (b).

16. The method of claim 1, wherein the oligonucleotides are phophorothioate oligonucleotides.

17. The method of claim 16, wherein the therapeutic oligonucleotide is a 5 to 50 mer.

18. A method for reducing the in vivo toxicity of a therapeutic oligonucleotide which tends to form multimeric aggregates comprising the step of treating a composition comprising the therapeutic oligonucleotide in multimeric aggregate form to convert substantially all of the therapeutic oligonucleotide to a monomeric form or to substantially prevent the formation of multimeric aggregates.

19. The method of claim 18, wherein the composition comprising the therapeutic oligonucleotide is treated by heating the composition.

20. The method of claim 19, wherein the composition is heated to a temperature of from 60 to 90° C. for a period of 3 to 60 minutes.

21. The method of claim 18, wherein the therapeutic composition is treated by addition of a chemical additive effective to substantially prevent aggregation.

22. The method of claim 18, wherein the therapeutic oligonucleotide is a 5 to 50 mer.

23. A composition comprising therapeutic oligonucleotides which tend to form multimeric aggregates in solution, said composition having been treated such that substantially all of the therapeutic oligonucleotides are in monomeric form.

24. The composition of claim 23, wherein the composition comprises a chemical additive effective to substantially prevent aggregation of the therapeutic oligonucleotides.

25. The composition of claim 24, wherein the chemical additive is mannitol or sucrose.

26. The composition of claim 23, wherein the oligonucleotides are phosphorothioate oligonucleotides.


SEQUENCE LISTING (2 sequences)

SEQ ID NO: 1 - DNA, 15 bp, Artificial
  antisense against c-myc RNA
aacgttgaggggcat

SEQ ID NO: 2 - DNA, 20 bp, Artificial
  antisense for ICAM-1 mRNA
gcccaagctggcatccgtca